(19) United States
(12) Patent Application Publication
(10) Pub. No.: US 2018/0140869 A1
(43) Pub. Date: May 24, 2018

(54) DETERMINATION OF POSITIONS OF OBJECTS, SUCH AS BRACHYTHERAPY SEEDS

(71) Applicant: KONINKLIJKE PHILIPS N.V., EINDHOVEN (NL)

(72) Inventors: Petrus Carolus Maria Frissen, Beek (NL); Willem Potze, Geldrop (NL); Aditya Mehendale, Geldrop (NL); Jacek Lukasz Kustra, Eindhoven (NL); Peter Prinsen, Eindhoven (NL); Andreas Goedicke, Aachen (NL)

(73) Assignee: KONINKLIJKE PHILIPS N.V., EINDHOVEN (NL)

(21) Appl. No.: 15/571,938

(22) PCT Filed: May 30, 2016
(86) PCT No.: PCT/EP2016/062098

§ 371 Date: Nov. 6, 2017

(30) Foreign Application Priority Data
Jun. 4, 2015 (EP) 15170636.3

(51) Int. Cl. A61N 5/10

(52) U.S. Cl. CPC A61N 5/1027; A61B 2090/3958; A61N 5/1049

(57) ABSTRACT

The invention relates to a device for determining positions of objects (1i) positioned in a measurement area, wherein the objects (1i) are capable of at least temporarily generating a magnetic field. The device comprises magnetometers (3i) arranged at a plurality of locations in a vicinity of the measurement area for locally measuring the magnetic field generated by the objects (1i), and an evaluation unit (5) coupled to the magnetometers (3i), the evaluation unit (5) being configured to determine the positions of the objects (1i) on the basis of the magnetic field measurements by the magnetometers (3i). The objects (1i) may be included in a human or animal body and may particularly be brachytherapy seeds.

### FIELD OF THE INVENTION

The invention relates to the determination of positions of objects. More specifically, the invention is related to a device and a method for determining positions of objects positioned in a measurement area. The objects may be included in a container, which may particularly be a human or animal body. The objects may particularly be brachytherapy seeds.

### BACKGROUND OF THE INVENTION

The determination of positions of certain objects is of interest particularly in medical applications. One such application is the so-called brachytherapy applied for the treatment of prostate cancer and other types of cancer. Brachytherapy involves the implantation of radioactive particles—so-called seeds—into the prostate or another area requiring treatment. The seeds emit ionizing radiation to treat the surrounding tissue with the main goal to destroy the cancer cells in this tissue.

In order to locate brachytherapy seeds within the body, WO 03/15864 A2 suggests to include magnetized or magnetizable material in the seeds. An oscillating magnetic field is applied to the seeds, which causes the seeds to vibrate. The vibrations are detected using Doppler ultrasound in order to determine the positions of the seeds. On the basis of the determined positions, it can be judged whether the seeds are placed accurately to treat the cancerous tissue or whether an implantation of additional seeds is necessary.

This method for locating brachytherapy seeds has the disadvantage that an ultrasound sensor needs to be affixed on or in the patient's body for carrying out the ultrasound measurements and the measurements usually have to be performed by a person skilled in ultrasound measurements. This makes the measurements inconvenient for the patient and usually requires that the measurements are carried out by skilled staff in a corresponding facility, such as a hospital or a doctor's practice. Further, the propagation of ultrasound through the patient's body depends on the composition of the tissue to be traversed and certain tissue may cause distortions of the propagation. This can lead to a deterioration of the image quality.

### SUMMARY OF THE INVENTION

It is an object of the present invention to allow for a more convenient and reliable localization of objects, such as brachytherapy seeds.

In a first aspect of the invention, a device for determining positions of objects positioned in a measurement area is provided. The objects include a magnetizable material and are capable of at least temporarily generating a magnetic field. The device comprises at least one electromagnetic coil for generating an excitation magnetic field to thereby magnetize the objects when they are positioned in the measurement area. The device further comprises magnetometers arranged at a plurality of locations in a vicinity of the measurement area for locally measuring the magnetic field generated by the objects, and the device comprises an evaluation unit coupled to the magnetometers, the evaluation unit being configured to determine the positions of the objects on the basis of the magnetic field measurements by the magnetometers. Thus, the objects can be excited to generate the magnet field during the measurement by magnetizing the objects using the electromagnetic coil. When no measurement is carried out, the objects may not generate a magnetic field. The measurement of the device is done by directly detecting the eddy currents in any arbitrary conductive material in the object and does not need the object to contain a coil, capacitor or any other circuit.

Since the device determines the positions of the objects on the basis of the measurement of the magnetic field generated by the objects at the locations of the magnetometers, it is possible to determine the object positions from a greater distance. The magnetometers do only have to be arranged in the vicinity of the objects. Further, the measurements can be performed without special skills (as e.g. required for carrying out ultrasound measurements). In case the objects are brachytherapy seeds, it is even possible that a patient performs the measurements himself at home.

The objects may be included in a container, which may particularly be a human or animal body. In this case, it is possible to non-invasively determine the object positions. Moreover, it is not necessary to affix any sensor directly to the container.

In one embodiment, the evaluation unit is configured to control the electromagnetic coil to generate the excitation magnetic field with an alternating magnitude, and that the evaluation unit is further configured to determine the positions of the objects on the basis of magnetic field measurements by the magnetometers at a time when the excitation magnetic field has a minimum magnitude. The minimum magnitude may correspond to zero or a small value. By using an alternating excitation magnetic field, it is possible to discriminate the excitation magnetic field from the induced magnetic field which is generated by the objects and which is used for determining the positions of the objects.

In one embodiment, the evaluation unit is configured to calculate values of the magnetic field generated by the objects at the locations of the magnetometers on the basis of estimated positions of the objects in a model calculation and to determine the positions of the objects by at least approximately minimizing a difference between the calculated values of the magnetic field and values measured by the magnetometers.

In the model calculation, the objects may be regarded as magnetic dipoles, and the magnetic field generated by the objects may be approximated as a superposition of magnetic fields of the magnetic dipoles. Such a model calculation leads to a non-linear system of equations including the positions of the objects as unknowns. By solving the system of equations, the positions of the objects can be determined.

The objects may particularly be brachytherapy seeds. In this case, the device allows for monitoring the positions of the brachytherapy seeds after they have been implanted into the body of a brachytherapy patient. This may also include the monitoring of the relative positions of the seeds by consecutive determinations of the positions of the seeds. Hereby, changes of the relative seeds positions may particularly be determined. Such changes of the relative seed positions are an indicator of a possible tumor (re-) growth. Thus, the detection of such changes allows for an early recognition of situations in which the tumor grows (again) so that it is made possible to consider appropriate treatment options already at an early stage.

One complication with respect to the monitoring of the relative seeds position is that the body is usually not identically positioned in the measurement area during different consecutive measurements of the magnetic field generated by the objects. Therefore, it is usually not possible to detect changes of the relative object positions by directly comparing the determined position of the objects with each other.

In this regard, one embodiment provides that the evaluation unit is configured to calculate a first value of a metric for first determined positions of the objects and to compare the first value with a second value of the metric calculated for second determined positions of the objects, the metric being indicative of relative positions of the objects. The latter does particularly mean that the value of the metric changes when the relative positions of the objects change.

In order to allow for a comparison of values of the metric calculated for object patterns which are determined on the basis of different positions of the body within the measurement area, the metric is preferably translation-invariant and rotation-invariant. One such metric, which may be used within the scope of the present invention, is a Hausdorff measure.

Further, in order to allow for a monitoring of the relative object positions over time, the first determined positions may be determined at a first point in time for a body and the second determined positions are determined at a second point in time for the same body. Since a change of the value of the metric (which is indicative of a change of the relative positions of the objects) is indicative of tumor growth, when the objects are brachytherapy seeds, the evaluation unit may be configured to control the localization device to output an alarm, if a difference between the first and second value of the metric exceeds a threshold.

In a further embodiment, the evaluation unit is configured to assign to each position of first determined positions of the objects one position of second determined positions of the objects. In a related embodiment, the evaluation unit is configured to carry out this assignment on the basis of a k-nearest neighbor algorithm. By means of such an algorithm, it is possible to assign object positions to each other which have been determined on the basis of consecutive measurements in which the body is positioned differently within the measurement area.

Further, a related embodiment comprises that the evaluation unit is configured to compare at least one distance between the first determined positions with a distance between the second determined positions assigned to the first determined positions. Here, the first determined positions may be determined at a first point in time for a body and the second determined positions may be determined at a second point in time for the same body. Moreover, the evaluation unit may be configured to control the device to output an alarm, if a difference between the at least one distance between the first determined positions and the distance between the second determined positions assigned to the first determined positions exceeds a threshold.

In a further aspect, the invention provides a method for determining positions of objects within a body positioned in a measurement area. The objects are capable of at least temporarily generating a magnetic field. The method comprises the steps of:
magnetometers locally measuring the magnetic field generated by the objects at a plurality of locations in a vicinity of the measurement area, and
an evaluation unit coupled to the magnetometers determining the positions of the objects on the basis of the magnetic field measurements by the magnetometers.
   It shall be understood that the device of claim 1 and the method of claim 15 have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily components of one embodiment of a device for determining the positions of objects in one embodiment,

FIG. 2 shows schematically and exemplarily one embodiment of an array of magnetometers,

FIG. 3a shows schematically and exemplarily an excitation magnetic field generated by a rectangular electromagnetic excitation coil, and

FIG. 3b shows schematically and exemplarily an induced magnetic field generated by an object in reaction to the excitation magnetic field.

### DETAILED DESCRIPTION OF EMBODIMENTS

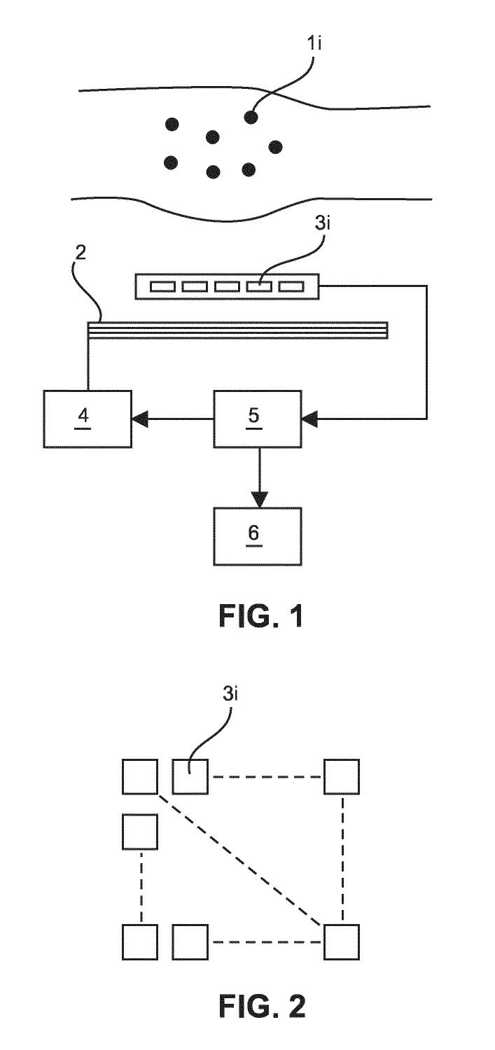

FIG. 1 schematically and exemplarily shows components of a localization device for determining positions of objects. In general, the localization device may be used to determine the positions of any magnetizable objects, particularly within a magnetically transparent container or at other inaccessible locations. In the illustrated embodiment, the objects are so called brachytherapy seeds 1i for treating prostate cancer or other types of cancer of a patient. Such seeds 1i are configured as small objects which particularly comprise a radioactive material. As already explained above, they are implanted into the organ or other area of the patient body to be treated and emit radioactive radiation to kill the cells of the surrounding tissue, particularly including the cancer cells. The implantation of the seeds 1i is usually made using implantation catheters, where the seeds 1i are placed via these catheters at their desired locations.

In addition to the localization of the objects, the localization device may also be capable of comparing determined positions of the objects with previously determined positions particularly in order to estimate whether the relative positions of the objects have been changed. Such an estimation of changes of the relative positions of the objects is especially advantageous in the aforementioned application in which the positions of brachytherapy seeds 1i are determined. So, the changes of the relative positions of the implanted seeds 1i in the prostate or other organ can be an indicator of tumor (re-)growth. Therefore, the monitoring of changes of the relative seed positions allows for an early detection of deteriorations of the patient's health status so that physicians can timely consider appropriate treatment options for the patient at an early stage.

In order to determine the positions of the seeds 1i using the localization device, the seeds 1i include a magnetized and/or magnetizable material. Thus, the seeds 1i may be composed of a material composition comprising at least the aforementioned radioactive material and the magnetized and/or magnetizable material. Here, it is in principle possible to use any magnetized and/or magnetizable material known to a person skilled in the art. Examples of materials which may be used are electrically conductive materials like copper, silver and gold or permeable materials like ferrite-ceramic coated in conductive materials. In one embodiment, these materials may be included within the seeds 1i and the seeds 1i may have an outer shell which is made of a biocompatible material, such as, for example, titanium. Hereby, biocompatibility of the seeds 1i can be ensured.

In case the seeds 1i include a magnetized material, they generate a magnetic field which is measured by means of the localization device in a way described in more detail below in order to determine the positions of the seeds 1i. In order to determine the positions of seeds 1i including a magnetizable material, which is not magnetized before the measurement, the localization device includes at least one electromagnetic excitation coil 2, which generate(s) a magnetic field for magnetizing the seeds 1i (in the following, it is assumed that one excitation coil 2 is used). After magnetization, the seeds 1i generate a magnetic field which is measured in order to determine their positions.

The excitation coil 2 is a conventional electromagnetic coil which is arranged in the vicinity of a measurement area, i.e. the volume in which the part of the patient body accommodating the seeds 1i is placed during the measurement using the localization device. The excitation coil 2 is coupled to an adjustable power source 4 which is configured to drive a current flowing through the excitation coil 2. Due to the current flowing through the excitation coil 2, a magnetic field—which is also referred to as excitation magnetic field herein—is generated by the excitation coil 2. In order for the excitation magnetic field to magnetize the seeds 1i, the excitation coil 2 is arranged in the vicinity of the measurement area in such a way that the strength of the excitation magnetic field in the (complete) measurement area is sufficiently high. For this purpose, a relative large single excitation coil 2 may be used. However, it is likewise possible to use multiple excitation coils 2 (which may be smaller) arranged in the vicinity of the measurement area in order to achieve a sufficiently high magnetic field strength throughout the measurement area.

The magnetic excitation may have arbitrary directions in the measurement area. In particular, it is not required that the magnetic excitation field is homogeneous within the area or has another particular form. Therefore, it is generally not necessary to configure the excitation coil 2 (or the plural excitation coils 2, if a plurality of excitation coils 2 is used) in a particular way in order to ensure the generation of an excitation magnetic field having a certain form. Hereby, the setup of the localization device is facilitated. The measurement according to the invention is based on directly detecting the eddy currents in any arbitrary conductive material in the seed and does not need the seed to contain a coil, capacitor or any other circuit.

For measuring the magnetic field generated by the seeds 1i, the localization device 1 further includes a plurality of magnetometers 3i. Each magnetometer 3i is configured to locally measure the strength of the magnetic field at its position. For this purpose, the magnetometers 3i may be configured in any suitable manner known to a person skilled in the art. For example, the magnetometers 3i may include hall sensors; however, other configurations are likewise possible. The magnetometers 3i may be configured as three-axis magnetometers measuring the magnetic field strength along three perpendicular directions. However, it is likewise possible to use one-axis or two-axis magnetometers measuring the magnetic field along one axis or along two perpendicular axes.

The magnetometers 3i are arranged in the vicinity of the measurement area at predefined (i.e. known) positions. This does particularly mean that the magnetometers 3i are arranged at certain fixed positions with respect to a reference coordinate system, which is used in the determination of the positions of the seeds 1i. Further, the magnetometer axes are preferably aligned with three perpendicular directions, which may correspond to the x-, y,- and z-directions of the reference coordinate system.

In order to determine the positions of N seeds 1i, the magnetometers 3i are capable of performing 6N or more independent measurements. In order to achieve this, the localization device may include at least 2N three-axis magnetometers, 3N two-axis magnetometers or 6N one-axis magnetometers. Likewise, it is possible that the localization device 1 comprises a combination of magnetometers 3i measuring along different numbers of axis, when these magnetometers 3i together allow for performing at least 6N measurements. By using only or a large number of three-axis magnetometers 3i, the localization device can be made more compact.

When 6N or more independent measurements can be made, the number of measurements is equal to or larger than the number of degrees of freedom of the system of N seeds 1i as each seed 1i has 6 degrees of freedom. Thus, there is at least one measurement value per variable included in the equations for determining the seed positions so that a unique solution for the positions of all N seeds 1i can be estimated. The 6 degrees of freedom of one seed 1i include three translational degrees of freedom of the seed 1i (i.e. to the position within space). Further, they include three degrees of freedom for the magnetization of seed 1i. These three degrees of freedom correspond to the magnetic moment of the respective seed 1i which has a magnitude and a direction in space.

Usually, the number of implanted seeds 1i varies for different patients. Preferably, the number of magnetometers 3i included in the localization device is selected such that it is sufficient for the majority of patients. In the case of prostate cancer, typically 30-40 seeds 1i are implanted, so that the magnetometer configuration of the localization device 1 may allow for performing circa 300 measurements (which are sufficient for determining the positions of 50 seeds). However, the invention can be carried out for any number of seeds 1i and is not limited to this example.

In principle, the magnetometers 3i can be arranged at arbitrary positions in the vicinity of the measurement area as long as they are positioned sufficiently close to the measurement area to measure the strength of the magnetic field generated by the seeds 1i. In one specific implementation, the magnetometers 3i are arranged in a two-dimensional rectangular array as schematically illustrated in FIG. 2. Thus, the magnetometers 3i are arranged along straight columns and rows arranged perpendicular to the columns. This implementation allows for a compact arrangement of the magnetometers 3i, and such an array can easily be manufactured. Further, the excitation coil 2 and the magnetometer array may be arranged on the same side of the measurement area. Thus, the magnetometer array and the excitation coil 2 can be arranged in a compact housing of the localization device, which may be positioned at one side of the measurement area.

In further embodiments, the magnetometers 3i may be included in two or more arrays of the aforementioned type. These arrays may be positioned at different sides of the measurement area. Thus, they may be arranged at a certain angle relative to each other and/or parallel to each on opposing sides of the measurement area. These embodiments do likewise allow for an easy installation of the magnetometers 3i. Moreover, these embodiments have the advantage that the values of the magnetic field generated by the seeds 1i can be measured at different positions having a greater distance to each other. Hereby, the measurement can be improved particularly in case this magnetic field is substantially homogenous in one or more limited area(s) and has a different form at more distant locations.

In a further possible implementation, the magnetometers 3i are arranged in a three-dimensional grid. Compared with the arrangement of the magnetometers 3i in a two-dimensional grid, this implementation may allow for an even more compact arrangement of the magnetometers 3i within the localization device.

However, as explained above, the magnetometers 3i can, in principle, be arranged at arbitrary known locations. Therefore, it is also possible, for example, that the magnetometers 3i may be arranged along a curved track in the vicinity of the measurement area.

The magnetometers 3i and the power source 4 driving the excitation coil 2 are coupled to an evaluation unit 5, which is particularly configured to evaluate the magnetometer measurements and to control the power source 4. The evaluation unit 5 may be configured as processer unit comprising a microprocessor for executing computer programs and a memory for storing the computer programs and further data.

Further, the localization device preferably comprises an output unit 6 for outputting the results of the evaluations made in the evaluation unit 5. Preferably, the output unit 6 may comprise a display device which is optionally capable of visualizing the determined seed pattern. However, displaying a graphical representation of the seed pattern requires a relatively large display unit with a sufficiently high resolution. In order to allow for a more compact and less complex design of the localization device, the output unit 6 may not be enabled to output a graphical representation of the seed pattern but may rather only output the result of the aforementioned estimation of the changes of the relative seed positions (as further explained below).

In one embodiment, the localization device including the aforementioned components may be configured as a portable device, which patients may use at home for determining positions of the brachytherapy seeds 1i and for monitoring changes of the relative seed positions. In this embodiment, the magnetometers 3i, the excitation coil 2 and the power supply 4 associated to the excitation coil 2 may be integrated into a housing which may be attached to a stand or other suitable support means so that it can be positioned next to a bed, chair or the like. Hereby, it is possible for the patient to operate the localization device to perform the determination of the seed positions at home, e.g. while lying in his bed or sitting on a chair so that the area to be examined (i.e. the organ including the seeds 1i) can be held in a fixed position during the measurement process. The evaluation unit 5 and the output unit 6 connected thereto may be integrated into the same housing as the magnetometers 3i and the excitation coil 2 in one implementation. As an alternative, the evaluation unit 5 and/or the output unit 6 may be arranged in a separate housing, which may be connected to the housing including the magnetometers 3i and the excitation coil 2 by a wired connection, for example.

In further implementations, the localization device may be configured as a portable or stationary device which is not used at home but in a hospital or another health care facility. In this case, the localization device may not be operated by the patient. Rather, a physician examining the patient may operate the localization device.

In order to determine the seed positions using the localization device, the region of the patient's body including the seeds 1i is arranged within the measurement area surrounded by the magnetometers 3i and the excitation coil 2.

In case, the seeds 1i are not magnetized, the excitation coil 2 is operated to create an induced magnetic field generated by the seeds 1i. For this purpose, an excitation magnetic field with an alternating strength is preferably generated by the excitation coil 2. This does particularly mean that the strength of the excitation magnetic field varies with a predetermined frequency between a maximum value and a minimum value. In order to achieve this, the evaluation unit 5 controls the power supply 4 to provide a time-varying current to the excitation coil 2. The strength of the current varies with a predetermined frequency between the maximum value and the minimum value, which may be zero or larger, to thereby generate an excitation magnetic field varying with the same frequency. In one embodiment, the direction of the current and, thus, the direction of the excitation magnetic field is not changed in the successive periods of the course of the current in time. However, in a further embodiment, the direction of the current and, consequently, the direction of the excitation magnetic field changes from one period to the next. The amplitude of the current may be constant, or may likewise differ in the periods in which the current flows in one direction on the one hand and the periods in which the current flows in the opposite direction on the other hand.

When exposed to the excitation magnetic field generated by the excitation coil 2, the magnetizable material included in the seeds 1i is magnetized so that an induced magnetic field is generated by the seeds 1i. Since the macroscopic magnetization of the magnetizable material is not built up instantaneously, the induced magnetic field is built up with a time delay with respect to the excitation magnetic field. After the macroscopic magnetization is built up, it vanishes again with a certain decay rate. When the seeds 1i are exposed to the alternating excitation field, an induced magnetic field is thus created, which does likewise alternate with a certain frequency.

In case the alternating excitation magnetic field has the same amplitude and direction in each period of the current flowing through the excitation coil 2, an (approximately) identical induced magnetic field is created in each period. If the direction of the excitation magnetic field changes from one period to the next, the direction of the induced magnetic field does likewise change. And even if the amplitude of the excitation magnetic field remains constant in each period, the magnitude of the induced magnetic field does likewise change from one period to the next in case the seeds 1i are not spherical (which is the usual case). The reason for this behavior is that the direction of the induced magnetic field created by each seed 1i corresponds to the direction of the excitation magnetic field. The magnitude of the induced magnetic field depends on the shape of the seed 1i and varies with the direction of the excitation magnetic field in case the seed 1i is not spherical.

The time delay for building up a macroscopic magnetization of the seeds 1i does particularly depend on the magnetizable material included in the seeds 1i. Within the scope of the present application, the magnetizable material and the frequency of the alternating excitation magnetic field are preferably selected such that the frequency of the induced magnetic field approximately corresponds to the frequency of the excitation magnetic field and such that the maximum strength of the induced magnetic field occurs when the strength of the magnetic excitation field is minimal.

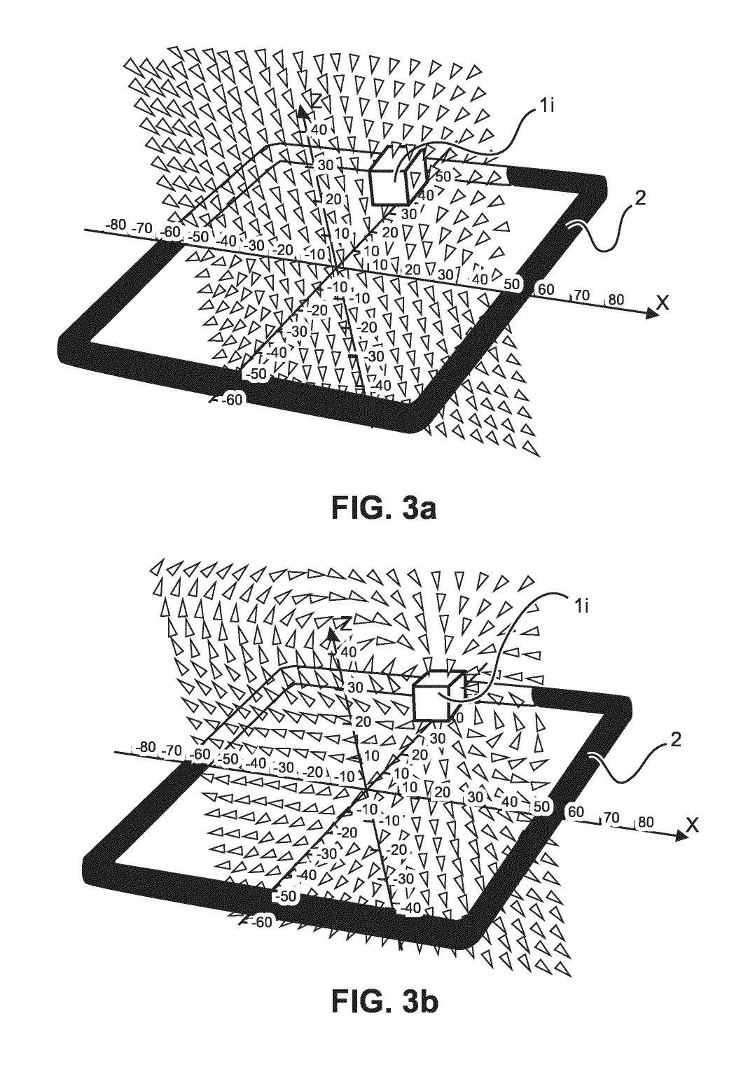

By way of example, this is schematically illustrated in FIGS. 3a and 3b for a single seed 1i having a cubic form (which is usually not the case): Here, FIG. 3a shows the magnetic field created by a rectangular excitation coil 2 at a point in time when the magnetic excitation field has its maximum strength and when the induced magnetic field is zero. FIG. 3b shows the induced magnetic field generated by the seed 1i at a later point in time, when the strength of the magnetic excitation field is zero.

The positions of the seeds 1i within the patient's body are determined on the basis of the induced magnetic field generated by the seeds 1i. When the excitation coil 2 is operated to generate an alternating magnetic excitation field as explained above, this induced magnetic field can be measured while the strength of the magnetic excitation field is minimal. Thus, in order to measure the induced magnetic field generated by the seeds 1i, the evaluation unit 5 reads measurement values measured by the magnetometers 3i in one or more time windows corresponding to the times when the strength of the excitation magnetic field or the strength of the current flowing through the excitation coil 2 is approximately zero. In one corresponding time window, the measurements performed by the magnetometers 3i yield values for the local strength of the magnetic field at the positions at which the magnetometers 3i are located with respect to the measurement axes of the magnetometers 3i. By performing measurements in plural consecutive corresponding time windows, plural measurement values can be captured for each position and axis. For each position and axis, the evaluation unit 5 may generate one value from the captured measurements values and this value may be used in the subsequent determination of the positions of the seeds 1i. For instance, this value may be a mean value of the captures measurement values.

In case the direction of the alternating excitation magnetic field does not change, one value may be generated for each position and axis from the measurement values captured in each period of the current flowing through the excitation coil 2. On the basis of the (single) values for all positions and axes, the positions of the seeds 1i may be determined. If the direction of the alternating excitation magnetic field changes from one period to the next, the evaluation may be made separately for first periods in which the excitation magnetic field has one direction and second periods in which the excitation magnetic field has the opposite direction. This means that one first value is determined for each position and axis on the basis of the measurements in the first periods and one second value is determined for each position and axis on the basis of the measurements in the second periods. The positions of the seeds 1i are then estimated on the basis of the first values and on the basis of the second values separately so that two (intermediate) estimates for the positions of each seed 1i are produced. The final estimate for the position of each seed 1i may then be made using the two intermediate estimates, e.g. by calculating the mean value of the two intermediate estimates. Thus, due to the larger number of different measurements, the determination of the of the seed positions is based on a “better statistic” when the excitation magnetic field changes directions so that the seed positions can be detected more reliably.

In case the seeds 1i include a magnetized material, the magnetic field generated by the seeds 1i can be directly measured using the magnetometers 3i without having to generate an excitation magnetic field. In this case, the evaluation unit 5 reads the measurement values for the local strength of the magnetic field created by the seeds 1i when the seeds 1i are placed in the measurement area of the localization device.

Thus, in both cases (i.e. when the seeds 1i are excited to generate an induced magnetic field and when the seeds 1i include a magnetized material generating a magnetic field) the evaluation unit 5 acquires from the magnetometers 3i a number of measurement values \vec{B}ₘ(\vec{x}ₘ) for the local strengths of a magnetic field generated by seeds 1i at the positions \vec{x}ₘ of the magnetometers 3i (where not all components of \vec{B}ₘ(\vec{x}ₘ) are measured in case the respective magnetometer is not configured as a three-axes magnetometer). On the basis of these measurements values, the evaluation unit 5 determines the positions of the seeds 1i. For this purpose, the evaluation unit 5 may calculate the magnetic field generated at the positions \vec{x}ₘ by the seeds 1i based on estimated positions and orientations of the seeds 1i in a model calculation and may determine the estimated positions and orientations of the seeds 1i such that a difference between the calculated magnetic field and the measurement values is minimized. The model calculation is preferably carried out on the basis of the Maxwell equations. Thus, the minimization of the difference between the calculated magnetic field and the (locally) measured magnetic field corresponds to a numerically determined solution of the inverse Maxwell problem.

In the model calculation, each seed 1i is regarded as a magnetic dipole. Thus, the induced magnetic field generated by each seed 1i is modeled by the equation describing the magnetic field of a magnetic dipole:

$${\vec{B}}_{n} ( \vec{y} ) = \frac{{μ}_{0}}{4 π} ( \frac{3 ( \vec{y} - {\vec{x}}_{n} ) ( {\vec{m}}_{n} \cdot ( \vec{y} - {\vec{x}}_{n} ) )}{{\vert \vec{y} - {\vec{x}}_{n} \vert}^{5}} - \frac{{\vec{m}}_{n}}{{\vert \vec{y} - {\vec{x}}_{n} \vert}^{3}} )$$

Here, \vec{B}ₙ (\vec{y}) denotes the vector of the magnetic field (B field) generated by the seed n at the position \vec{y}, \vec{x}ₙ denotes the position of the seed n and \vec{m}ₙ denotes the magnetic dipole moment of the seed n.

The induced magnetic field \vec{B}ₜₒₜ(\vec{y}) generated by all seeds 1i at the position \vec{y} results from a superposition of the magnetic fields generated by the individual seeds 1i and is given by \vec{B}ₜₒₜ (\vec{y})=Σₙ \vec{B}ₙ (\vec{y}), where the sum is calculated over all seeds 1i.

Now, each magnetometer 3i of the localization device 1 measures one or more components of the field \vec{B}ₜₒₜ(\vec{y}ₖ) at the position \vec{y}ₖ of the respective magnetometer 3i, and the measurements carried out by all magnetometers 3i yield a number of values B_{tot,l}(\vec{y}ₖ), where B_{tot,l} denotes the l-component of the field \vec{B}ₜₒₜ (i.e. the x-, y- or z-component). Thus, the measurements allow for establishing a (non-linear) system of equations in which each equation has the form

$${B}_{tot , l} ( {\vec{y}}_{k} ) = \frac{{μ}_{0}}{4 π} {\sum }_{n} ( \frac{3 ( {y}_{l} - {x}_{n , l} ) ( {\vec{m}}_{n} \cdot ( \vec{y} - {\vec{x}}_{n} ) )}{{\vert \vec{y} - {\vec{x}}_{n} \vert}^{5}} - \frac{{m}_{n , l}}{{\vert \vec{y} - {\vec{x}}_{n} \vert}^{3}} ) .$$

Here, the subscript l does again denote the l-component of the respective parameter.

As unknowns, the system of equations includes (on the right-hand side) the components of the positions \vec{x}ₙ of the seeds 1i, which are to be estimated, and the components of the magnetic moments \vec{m}ₙ of the seeds 1i.

In order to estimate the positions of the seeds 1i, the evaluation unit 5 of the localization device 1 establishes a system of equations of the aforementioned type. The system includes one equation for each value B_{tot,l}(\vec{y}ₖ) determined on the basis of the measurements in the way described above. The sums on the right-hand side of the equations are established on the basis of the number of seeds 1i, which is provided to the evaluation unit 5 in addition to the measured values for the magnetic field, and which is input into the localization device by the user in one embodiment.

Then, the evaluation unit 5 determines a solution of the system of equations for the unknown positions \vec{x}ₙ of the seeds 1i and their magnetic moments \vec{m}ₙ. Preferably, the solution of the system of equations is determined using a numerical algorithm. In this regard, any suitable algorithm known to a person skilled in the art may be applied. One example of such an algorithm is the well-known Gauss-Newton algorithm, which allows for approximating a solution of the system of equations in a least-square sense.

After having estimated the positions of the seeds 1i, the evaluation unit 5 may control the output unit 6 to show a graphical representation of the seed pattern in accordance with the determined positions, when the output unit 6 is capable to display such a graphical representation. Thus, it possible for the user of the localization device 1 (e.g. the patient or a physician operating the localization device 1) to visually inspect the estimated seed pattern.

Moreover, the evaluation unit 5 may perform a further evaluation of the determined seed positions which allows for comparing the relative seed positions determined at consecutive points in time. Thus, it is made possible to monitor the relative seed position over a certain period of time such as several days, weeks or months, for example, on the basis of the results for the positions determined based on two or more measurements during such period of time. As explained above, such monitoring allows for detecting changes of the seed pattern, which may particularly be indicative of a tumor growth.

In this regard, the relative position of the patient's body and the magnetometers 3i is usually different during the measurements carried out for determining the seed positions. Thus, it is usually not possible to directly compare positions of the seeds 1i determined on the basis of one measurement with the positions determined on the basis of a previous measurement.

Therefore, the evaluation unit 5 may calculate a metric for the positions determined on the basis of each measurement and may compare the calculated values of the metric in order to determine a potential change of the relative seed positions. Such a metric is a parameter assigned to the positions of the seeds 1i, which is influenced by the relative seeds positions so that the metric changes, when the relative positions of the seeds change. Moreover, the metric is preferably selected such that it does not change when the complete seed pattern is shifted and/or rotated. Thus, a translation-invariant and rotation-invariant metric is selected.

An example of such a metric is the Hausdorff measure, which is as such known to a person skilled in the art. In the present application, the Hausdorff measure H^{d} (S) for the set S of the N seeds with positions \vec{x}ᵢ (i=1, . . . , N) may be calculated as the minimum over all seeds of the sum of the d-th power of the distances between two seeds 1i. Thus, the Hausdorff measure H^{d} (S) is given by:

$${H}^{d} ( S ) = {min}_{⋃ \{ i , j \}} \{ \sum {\vert {\vec{x}}_{i} - {\vec{x}}_{j} \vert}^{d} \text{:} {{⋃}^{N}}_{i , j = 1 , i \neq j} \{ {\vec{x}}_{i} , {\vec{x}}_{j} ) ⊇ S \}$$

The parameter d may be set to 1 or 2, for example. However, any other suitable value may likewise be used for calculating the Hausdorff measure in the evaluation unit 5.

In further embodiments, other translation-invariant and rotation-invariant metrics may be used. Such metrics may be variations of the Hausdorff measure, for example. One example of such a variation is a measure M^{d} (S) calculated on the basis of the same parameters as the Hausdorff measure as follows:

$${M}^{d} ( S ) = {min}_{⋃ \{ i , j \}} \{ \sqrt[{}^{′} d]{\sum {\vert {\vec{x}}_{i} - {\vec{x}}_{j} \vert}^{d}} \text{:} {{⋃}^{N}}_{i , j = 1 , i \neq j} \{ {\vec{x}}_{i} , {\vec{x}}_{j} ) ⊇ S \}$$

A first value of a metric of the aforementioned type may be calculated from the positions of the seeds 1i determined on the basis of one measurement of the magnetic field generated by the seeds 1i. Then, the evaluation unit 5 may store the calculated value of the metric in a memory of the evaluation unit 5. After having determined the positions of the seeds 1i on the basis of a subsequent measurement of the magnetic field generated by the seeds 1i, the evaluation unit 5 may calculate a second value of the metric from the determined positions. A larger difference between the first and second value of the metric may be indicative of a change of the relative seed positions. Therefore, the evaluation unit 5 may compare the first value of the metric with the second value in order to determine whether the difference between both values exceeds a predefined threshold. If so, the evaluation unit 5 may control the output unit 6 to output an alarm to the user of the localization device.

If the localization device is used by the patient at home for monitoring the seed positions, the alarm may serve as an indication for the patient to consult a physician in order to further assess whether the tumor has grown.

As an alternative or as an addition to the comparison of different seed patterns on the basis of the metric, the evaluation unit 5 may use a k-nearest neighbors algorithm in order to facilitate the comparison of the seed positions determined based on different measurements. In this embodiment, the evaluation unit 5 may store the positions of the seeds 1i determined on the basis of a first measurement of the magnetic field generated by the seeds 1i. After having determined the seed positions on the basis of a subsequent second measurement of the magnetic field generated by the seeds 1i, the evaluation unit 5 may apply a k-nearest neighbor algorithm in order to assign to each determined seed position one of the stored seed positions derived from the first measurement. Here, the k-nearest neighbor algorithm, which is known to the person skilled in the art as such, ensures that the assignment of each newly determined seed position to one of the previously determined seed positions is made in accordance with the closest match between the newly determined seed pattern and the previously determined seed pattern.

After having assigned each newly determined seed position to one previously determined seed position, the evaluation unit 5 may compare the differences between the newly determined seed positions with the differences between the assigned previously determined seed positions. Such a comparison may be made for all pairs of seed positions in both seed patterns. If the evaluation unit 5 determines as a result of this comparison that the distance between newly determined seed positions differs from the distance between the assigned previously determined seed position by an amount exceeding a threshold, the evaluation unit 5 may control the output unit 6 to output a corresponding indication as explained above.

In the manner described, the localization device may be used to determine the positions of brachytherapy seeds 1i implanted into a patient's body. However, the invention is not limited to this particular application. Rather, the localization device may be used to determine positions of other objects within a body or other container, when these objects include a magnetized or magnetizable material. For instance, the localization device may be used to track the position of the implant catheters during the process of implanting brachytherapy seeds 1i. For this purpose, the catheter tips and/or other parts of the catheters may be provided with a magnetized and/or magnetizable material. In a similar manner, the positions of other catheters or probes within a human or animal body can be determined and tracked during a surgery or in the process of examinations.

Moreover, similar localization devices may be used in many non-medical applications. For instance, such a localization device may be used in order to determine the positions of metal objects in luggage or carried by person (i.e. within the person's clothes) at security checkpoints or the like. Moreover, a localization device of the aforementioned type may be used for determining the positions of magnetizable particles in fluids, for example. Further exemplary applications for a use of the invention are geological applications, where a localization device of the aforementioned kind may be used to determine the position of magnetizable objects in the ground.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

1. A device for determining positions of objects (1i) positioned in a measurement area, wherein the objects (1i) include a magnetizable material capable of at least temporarily generating a magnetic field and wherein the device comprises:
at least one electromagnetic coil (2) for generating an excitation magnetic field to thereby magnetize the objects (1i) when they are included in the measurement area,
magnetometers (3i) arranged at a plurality of locations in a vicinity of the measurement area for locally measuring values of the magnetic field generated by the objects (1i) due to their magnetization; and
an evaluation unit (5) coupled to the magnetometers (3i), the evaluation unit (5) being configured to determine the positions of the objects (1i) on the basis of the values of the magnetic field generated by the objects (1i) due to their magnetization measured by the magnetometers (3i).

2. The device as defined in claim 1, wherein the objects (1i) are included in a container positioned in the measurement area.

3. The device as defined in claim 1, wherein the evaluation unit is configured to control the electromagnetic coil to generate the excitation magnetic field with an alternating magnitude, and wherein the evaluation unit is further configured to determine the positions of the objects (1i) on the basis of magnetic field measurements by the magnetometers (3i) at a time when the excitation magnetic field has a minimum magnitude.

4. The device as defined in claim 1, wherein the evaluation unit is configured to calculate values of the magnetic field generated by the objects at the locations of the magnetometers (3i) on the basis of estimated positions of the objects (1i) in a model calculation and to determine the positions of the objects (1i) by at least approximately minimizing a difference between the calculated values of the magnetic field and values measured by the magnetometers (3i).

5. The device as defined in claim 4, wherein the objects (1i) are regarded as magnetic dipoles and the magnetic field generated by the objects (1i) is approximated as a superposition of magnetic fields of the magnetic dipoles in the model calculation.

6. The device as defined in claim 1, wherein the evaluation unit is configured to calculate a first value of a metric for first determined positions of the objects (1i) and to compare the first value with a second value of the metric calculated for second determined positions of the objects (1i), the metric being indicative of relative positions of the objects.

7. The device as defined in claim 6, wherein the metric is a Hausdorff measure.

8. The device as defined in claim 6, wherein the first determined positions are determined at a first point in time for a body and wherein the second determined positions are determined at a second point in time for the same body, and wherein the evaluation unit is configured to control the device to output an alarm, if a difference between the first and second value of the metric exceeds a threshold.

9. The device as defined in claim 1, wherein the evaluation unit is configured to assign to each position of first determined positions of the objects (1i) one position of second determined positions of the objects (1i).

10. The device as defined in claim 9, wherein the evaluation unit is configured to carry out the assignment on the basis of a k-nearest neighbors algorithm.

11. The device as defined in claim 9, wherein the evaluation unit is configured to compare at least one distance between the first determined positions with a distance between the second determined positions assigned to the first determined positions.

12. The device as defined in claim 11, wherein the first determined positions are determined at a first point in time for a body and wherein the second determined positions are determined at a second point in time for the same body, and wherein the evaluation unit is configured to control the device to output an alarm, if a difference between the at least one distance between the first determined positions and the distance between second determined positions assigned to the first determined positions exceeds a threshold.

13. The device as defined in claim 1, wherein the objects (1i) comprise brachytherapy seeds.

14. method for determining positions of objects (1i) positioned in a measurement area, wherein the objects (1i) include a magnetizable material capable of at least temporarily generating a magnetic field, the method comprising the steps of:
an electromagnetic coil (2) generating an excitation magnetic field to thereby magnetize the objects (1i) in the measurement area
magnetometers (3i) locally measuring values of the magnetic field generated by the objects (1i) due to their magnetization at a plurality of locations in a vicinity of the measurement area, and
an evaluation unit (5) coupled to the magnetometers (3i) determining the positions of the objects (1i) on the basis of the values of the magnetic field generated by the objects (1i) due to their magnetization measured by the magnetometers (3i).
